(19) United States
(12) Patent Application Publication
(10) Pub. No.: US 2021/0127719 A1
(43) Pub. Date: May 6, 2021

(54) AROMATIC MIXTURE FOR REDUCING THE ODOR OR TASTE OF BIOGENIC AMINES

(71) Applicant: SYMRISE AG, Holzminden (DE)

(72) Inventors: Johannes Kiefl, Holzminden (DE); Uwe Schaefer, Ottenstein (DE); Birgit Kohlenberg, Pegestorf (DE); Thomas Henze, Holzminden (DE); Stephanie Korte, Höxter (DE)

(21) Appl. No.: 16/471,409

(22) PCT Filed: Dec. 20, 2016
(86) PCT No.: PCT/EP2016/081939

§ 371 Date: Jun. 19, 2019

(51) Int. Cl. A23L 5/20; A23L 27/00; C11B 9/00; A61K 8/35; A61K 8/34; A61K 8/49; A61K 47/22; A61K 47/10

(52) U.S. Cl. CPC A23L 5/27; A23L 27/84; C11B 9/008; C11B 9/0076; C11B 9/0034; C11B 9/0053; A61K 47/10; A61K 8/35; A61K 8/34; A61K 8/498; A61K 8/494; A61K 47/22; C11B 9/0092

(57) ABSTRACT

The present invention relates to an aroma mixture or a foodstuff comprising an ionone or a related compound as well as an alkylated 1,4-pyrazine. Hereby, the unpleasant taste and odor of biogenic amines can be reduced.

The present invention relates to aroma compositions and foodstuffs.

The targeted production and use of aroma mixtures or foodstuffs of certain flavors is subject to constant research, particularly aiming to improve the unpleasant taste of certain foodstuffs.

In particular, the decomposition of nitrogen-containing ingredients such as proteins to biogenic amines and other odorants and flavors is known for its odor and taste, which are often perceived as unpleasant, which is why e.g. lemon juice is used in fish dishes. However, such additives are not always possible, and it is therefore a task to provide aroma mixtures or foodstuffs capable of improving the taste and/or odor of substances resulting from the decomposition of nitrogen-containing ingredients such as biogenic amines and volatile acids, or where the taste and/or odor of biogenic amines, which are perceived as detrimental, has been reduced.

This task is solved by an aroma mixture according to claim 1. Accordingly, an aroma mixture comprising a first component comprising a molecule selected from the group comprising molecules of the following structure (I) is proposed:

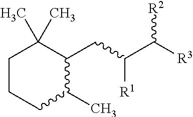

wherein [symbol] represents a single or double bond, wherein allenes are excluded,

R¹ is selected from hydrogen or methyl,

R² is O or OH, i.e. keto or hydroxy group; as well as

R³ is selected from methyl, ethyl, propyl, isopropyl or propenyl,

Molecules of the following structure (II):

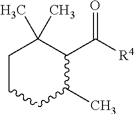

wherein represents [symbol] a single or double bond, wherein allenes or cumulene structures are excluded,

R⁴ is selected from hydrogen and propenyl,

2,5,5,8a-tetramethyl-7,8-dihydro-6H-chromene (cyclo ionone) 4,4,7a-trimethyl-6,7-dihydro-5H-benzofuran-2-one (dihydroactinidiolide) (E)-4-(1,5,5-trimethyl-7-oxabicyclo[4.1.0]heptan-6-yl)but-3-en-2-one (5,6-epoxy ionone) 2-(2,6,6-trimethylcyclohexen-1-yl) acetaldehyde ((β-homocyclocitral) (4E)-4-[(E)-but-2-enylidene]-3,5,5-trimethyl-cyclohex-2-en-1-one (Tabanon) (E)-1-(2,4,4-trimethylcyclohex-2-en-1-yl)but-2-en-1-one (α-isodamascone) 4,4,7-trimethyl-3,6,7,8-tetrahydronaphthalen-2-one

or mixtures thereof,

as well as a second component comprising an alkylated 1,4-pyrazine having one to four alkyl carbons.

Preferably, the first and/or second component consists predominantly of the respectively indicated molecules. The term “predominantly consist of” means in particular a share (in wt./wt.) of >95%, preferably ≥97% and most preferably ≥99%.

It should be noted that many of the molecules that fall particularly under the first component may be present as stereoisomers (e.g. cis/trans or enantiomers). Where not further specified, all possible stereoisomers are always comprised.

According to a preferred embodiment of the invention, when enantiomers are present, an excess of the enantiomer that is naturally predominantly present in nature is used.

Surprisingly, it has been shown that such an aroma mixture is able to reduce the taste and/or odor of biogenic amines in many applications or even to partially eliminate these completely.

According to a preferred embodiment, the aroma mixture thus comprises at least one biogenic amine.

The term “biogenic amines” refers in particular to amines and derivatives which may arise from protein degradation, in particular 2-phenylethylamine, cadaverine, histamine, putrescine, spermidine, spermine, tryptamine, tyramine, indole, skatole, methylamine, dimethylamine, trimethylamine, acetic acid, propionic acid, 2-methylbutyric acid, 3-methylbutyric acid, 4-methylpentanoic acid, diethylamine, ethylmethylamine and diethanolamine. Not all of these substances are usually referred to as amines; here a deviation from standard linguistic usage should explicitly apply.

Certain foodstuffs may contain high levels of these biogenic amines, such as dried anchovies (348 mg/kg), fish sauce (196-197 mg/kg), fermented vegetables (39.4-42.6 mg/kg), cheese (20.9-62 mg/kg), fish (26.8-31.2 mg/kg) and fermented sausage product (23.0-23.6 mg/kg), whereas traces are measured in other foodstuffs such as coffee, chocolate, juices, cereals and fruits (EFSA Journal 2011; 9(10):2393).

In most applications of the present invention, one or more of the following advantages may be observed or obtained:
the animalistic, fecal and fishy notes are diminished
the sweaty notes are diminished
the inherent odor of the base is enhanced reduced by the unpleasant notes
Improvement of the sensory qualities of e.g. fermented products (fish sauce, chocolate, beer etc.) and strongly processed products (special food from hydrolyzed protein etc.) by simple addition of one of the mentioned aroma mixtures.

Furthermore, the present object is solved by a foodstuff or foodstuff preparation which comprises at least one biogenic amine, additionally comprising the first and second components as described, wherein the first and second components are independently present in the foodstuff or foodstuff preparation in at least a concentration of ≥1 μg/kg.

It has been found in many applications that this is the lower limit for efficacy in foodstuff, particularly for the pyrazine component.

The present invention also refers to the use of an aroma mixture comprising the first and/or second component for reducing and/or masking the taste and/or odor of biogenic amines. It has been found that in many applications one component is sufficient to achieve such an effect.

The two inventive components are explained in more detail in the following:

First Component:

According to a preferred embodiment, the first component contains at least one molecule selected from the group comprising:

(1) (E)-4-(2,6,6-trimethylcyclohex-1-en-1-yl)but-3-en-2-one (β-ionone)

(2) (E)-4-(2,6,6-trimethylcyclohex-2-en-1-yl)but-3-en-2-one (α-ionone)

(3) (E)-4-(2,6,6-trimethylcyclohex-2-en-1-yl)but-3-en-2-ol (α-Ionol)

(4) (E)-4-(2,6,6-trimethylcyclohex-1-en-1-yl)but-3-en-2-ol (β-Ionol)

(5) 1-(2,2,6-trimethylcyclohexyl)pentan-3-ol (Madranol)

(6) 1-(2,2,6-trimethylcyclohexyl)hexan-3-ol (Timberol)

(7) 2,5,5,8a-tetramethyl-7,8-dihydro-6H-chromene (cyclo ionone)

(8) 4,4,7a-trimethyl-6,7-dihydro-5H-benzofuran-2-one (dihydroactinidiolide)

(9) (E)-4-(1,5,5-trimethyl-7-oxabicyclo[4.1.0]heptan-6-yl)but-3-en-2-one (5,6-epoxy-ionone)

(10) (E)-1-(2,6,6-trimethylcyclohex-3-en-1-yl)but-2-en-1-one (δ-damascone)

(11) (E)-1-(2,6,6-trimethylcyclohex-1-en-1-yl)but-2-en-1-one (β-damascone)

(12) (E)-1-(2,6,6-trimethylcyclohex-2-en-1-yl)but-2-en-1-one (α-damascone)

(13) (E)-1-(2,6,6-trimethylcyclohexa-1,3-dien-1-yl)but-2-en-1-one (β-damascenone)

(14) 2-(2,6,6-trimethylcyclohex-1-en-1-yl) acetaldehyde (β-homocyclocitral)

(15) 2,6,6-trimethylcyclohex-1-ene-1-carbaldehyde (β-cyclocitral)

(16) (E)-1-(2,4,4-trimethylcyclohex-2-en-1-yl)but-2-en-1-one (α-isodamascone)

(17) (4E)-4-[(E)-but-2-enylidene]-3,5,5-trimethyl-cyclohex-2-en-1-one (Tabanon)

(18) 4,4,7-trimethyl-3,6,7,8-tetrahydronaphthalen-2-one

or mixtures thereof, preferably the first component consists predominantly of these molecules. The structural formulas of the individual components are given below:

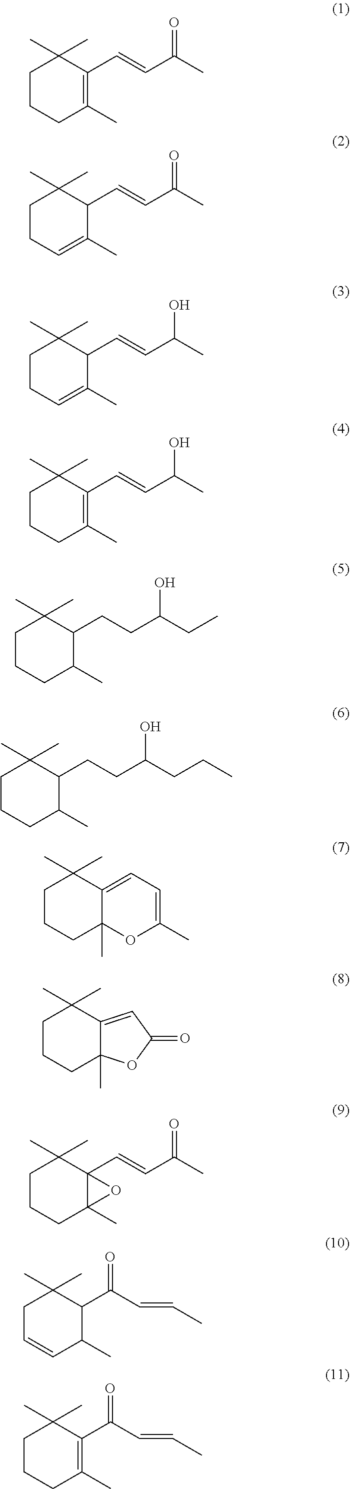

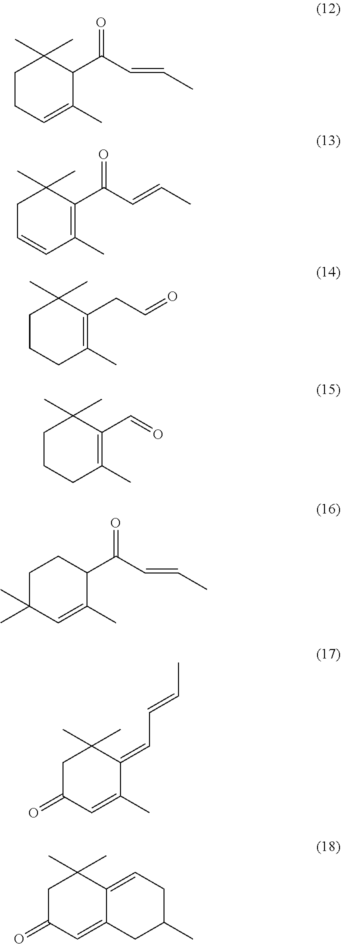

Second Component:

The second component comprises, according to a preferred embodiment of the invention, at least one molecule selected from the group comprising:

(19) 5-methyl-6,7-dihydro-5H-cyclopenta[b]pyrazine

(20) 2,3-diethyl-5-methyl-pyrazine

(21) 2-ethyl-3,5-dimethyl-pyrazine

(22) 3-ethyl-2,5-dimethyl-pyrazine

Or mixtures thereof, preferably consisting predominantly of these molecules. The structural formulas of these compounds are given below:

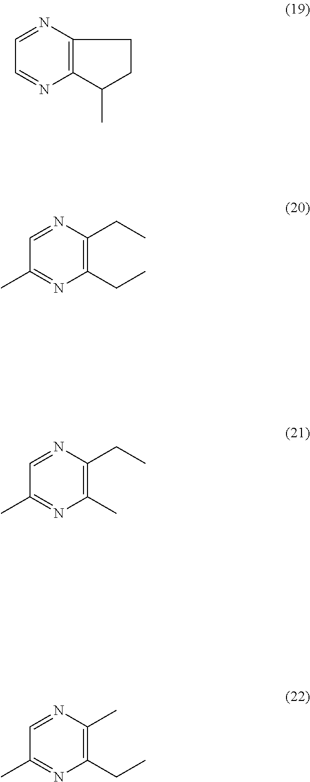

It should be noted that the compound (19) is a chiral compound, in which both the individual enantiomers and the racemate are preferred embodiments.

According to a preferred embodiment, the second component is added as a substance. Alternatively, or additionally, the second component is obtained by treating a precursor of the inventive aroma mixture or a composition containing a precursor of the inventive aroma mixture or by treating a precursor of the inventive foodstuff or foodstuff preparation, respectively. This shall also apply mutatis mutandis to the inventive use and the inventive foodstuff.

This can be done particularly in the sense of the Mailliard reaction from suitable amino acids and reducing sugars, as e.g. described in Hodge, J. E., Dehydrated Foods, Chemistry of Browning Reactions in Model Systems. J. Agric. Food Chem. 1953, 1, 928-943 or Maga, J. A., Pyrazine update. Food Rev. Int. 1992, 8, 479-558.

The inventive aroma mixture, the inventive foodstuff or foodstuff preparation, respectively, as well as the use according to the invention may comprise further ingredients.

According to a preferred embodiment of the invention, ingredients are selected from substances for changing or masking an unpleasant taste impression and/or for enhancing a pleasant taste impression and taste correctives, respectively, are preferably selected from the following group: 4-hydroxyflavanone e.g. as described in EP 1,258,200-B1, hesperetin as described in EP 2,368,442-B1 or EP 1,909,599-B1, hydroxybenzoic acid amides, such as 2,4-dihydroxybenzoic acid vanillylamide (in particular those described in WO 2006/024587), 4-hydroxydihydrochalcones (preferably as described in US 2008/0227867 A1 and WO 2007/107596), thereby in particular phloretin and davidigenin, hesperetin as disclosed in WO 2007/014879, or extracts from Rubus suavissimus as described in U.S. Provisional Application 61/333,435 (Symrise) and patent applications based thereon, 3,7′-dihydroxy-4′-methoxyflavan isomers as described in EP 2,253,226, phyllodulcin isomers or phyllodulcin-containing extracts as described in EP 2,298,084-B1, 1-(2,4-Dihydroxy-phenyl)-3-(3-hydroxy-4-methoxy-phenyl)-propan-1-one as described in EP 2,353,403-B1, neoisoflavonoids as described in EP 2,570,036-B1, pellitorin and derived aroma compositions as described in EP 2 008 530 A1, Vanillyl lignans, in particular as described in the European patent application with file number EP 2,517,574, neoisoflavonoids as described in EP 2,570,035-B1, Neohesperidin hydrochalcone, hesperetin dihydrochalcone, hesperidin dihydrochalcone, naringin dihydrochalcone, phloridzin, trilobatin, steviosides and/or rebaudiosides, in particular mixtures of various rebaudiosides as described in WO 2015 062,998, rubusosides as described in EP 2,386,211, mixtures of rubusoside isomers and homologues as described in the European application EP 14172306.4, mogrosides, abrusosides in and/or balansins as described in WO 2012 164,062 and mixtures of these substances.

Further ingredients can be selected from the list of volatile aromatics, e.g. acetaldehyde, acetamide, acetophenone, 2-acetylfuran, 2-acetylpyrrole, aldehyde C18 so-called, benzaldehyde, benzoic acid, 2(3H)-benzofuranone, benzonitrile, benzothiazole, benzyl alcohol, benzyl cyanide, benzyl methyl sulfide, beta-pinene, pyrocatechol, butanol, 2-butanone, 2E-butenal, 3-buten-2-one, butyric acid, butyl acetate, capric acid, gamma-caprolactone, caprylic acid, diacetyl, dihydrobovolid, 3,5-dihydroxy-6-methyl-2,3-dihydro-4-pyranone, dimethyldisulfide, 2,5-dimethyl-3-ethylpyrazine, 3,5-dimethyl-2-ethylpyrazine, 4,4-dimethyl-3-hydroxy-4,5-dihydro-2(3H)-furanone, 2,3-dimethylpyrazine, 2,5-dimethylpyrazine, 2,6-dimethylpyrazine, dimethyl sulfide, dimethyl sulfoxide, dimethyl trisulfide, acetic acid, ethyl-2-methyl butyrate, 5-ethyl-2-methyl-4-propionyloxy-3(2H)-furanone, ethyl-2-methyl thiocyanide, ethyl acetate, ethyl butyrate, 2-ethyl furan, ethyl furaneol, ethyl isovalerianate, ethyl phenyl acetate, ethyl propionate, ethylpyrazine, formamide, formanilide, furaneol, 2(5H)-furanone, furfuryl alcohol, gamma-heptalactone, 3-heptanone, heptanoic acid, hexanol, 2-hexene-1,4-olide, 2-indanone, indole, isoamyl alcohol, isobutyric acid, isobutyl cyanide, 3-isobutylhexahydropyrrolo[1,2-A]pyrazine-1-4-dione, isobutyraldehyde, 1-isopropyl-3-methylbenzene, isovaleraldehyde, isovaleric acid, para-cresol, lauric acid, limonene, methanethiol, methyl benzoate, 2-methylbutanal, 2-methylbutanol, 2-methylbutyric acid, 2-methyl-5-ethylpyrazine, 2-methyl-6-ethylpyrazine, 2-methylfuran, 6-methyl-5-hepten-2-one, 5-methyl-2,4-imidazolidinedione, 2-methyl-5-isopropylpyrazine, 4-methylphenylcyanide, 1-methylthio-3-pentanone, 3-methylthiopropanol, 4-methylvaleric acid, myristic acid, N-(3-methylbutyl)acetamide, 1,5E-octadien-3-ol, 1,5Z-octadien-3-ol, 2,5-octadien-1-ol, octanol, 2-octanol, 1-octen-3-ol, 2-oxopropanol, 3-pentanone, 1-penten-3-one, pentylpyrazine, 2-piperidone, phenol, phenylacetic acid amide, 2-phenylethylacetamide, 2-phenylethyl alcohol, 3-phenylpropanol, propanal, propanol, 2-propenal, propionamide, propionic acid, propiophenone, pyrazine, pyrrole, carbon disulfide, hydrogen sulfide, skatole, succinamide, sulfurole, thiophene, trimethylamine, 3,5,5-trimethylhexanoic acid, 2,3,5-trimethylpyrazine, valeric acid, 4-vinylguajacol, 4-vinylphenol, vinylpyrazine, and mixtures of these substances.

Other preferred ingredients are selected from the group consisting of reaction aromatics (Maillard products), extracts or essential oils of plants or parts of plants or fractions thereof, smoke flavorings or other aroma-providing preparations (e.g. protein [part] hydrolysates, grill flavorings, plant extracts, spices, spice preparations, vegetable varieties and/or vegetable preparations).

In particular, aromatics or their components are suitable, which are not comprised in the mixture and that a roasted, fleshy (in particular chicken, fish, seafood, beef, pork, lamb, sheep, goat), vegetable (in particular tomato, onion, garlic, celery, leek, mushrooms, egg plants, seaweed), a spicy (in particular black and white pepper, chili, paprika, cardamom, nutmeg, pimento, mustard and mustard products), roasted, yeasty, boiled, greasy, salty and/or pungent aroma impression and thus can strengthen the spicy impression.

The present invention also relates to ready-made products comprising an aroma mixture and/or a foodstuff according to the present invention. These can be foods and/or food supplements.

As far as the ready-made products are foods, they are, for example, bakery products such as bread, dry biscuits, cakes, other baked goods, confectionery (such as chocolates, chocolate bar products, other bar products, fruit gums, hard and soft caramels, chewing gum), alcoholic or non-alcoholic beverages (such as coffee, tea, iced tea, wine, wine-containing beverages, beer, beer-containing beverages, liqueurs, spirits, brandies, (carbonated) fruit-containing lemonades, (carbonated) isotonic beverages, (carbonated) soft drinks, nectars, spritzers, fruit and vegetable juices, fruit or vegetable juice preparations, instant beverages (such as instant cocoa drinks, instant tea drinks, instant coffee drinks, instant fruit drinks), meat products (such as ham, fresh sausage or raw sausage preparations), spiced or marinated fresh or cured meat products), eggs or egg products (dried egg, egg white, egg yolk), cereal products (such as breakfast cereals, muesli bars, pre-cooked ready-made rice products), dairy products (such as milk drinks, buttermilk drinks, dairy ice cream, yoghurt, kefir, cream cheese, soft cheese, hard cheese, dried milk powder, whey, whey drinks, butter, buttermilk, partly or wholly hydrolyzed products containing milk protein), products made from soya protein or other soya bean fractions (such as soya milk and products made from it, fruit drinks containing soya protein, preparations containing soya lecithin, fermented products such as tofu or tempeh or products made thereof), products from other vegetable protein sources such as oat protein drinks, fruit preparations (such as jams, sherbet, fruit sauces, fruit fillings), vegetable preparations (such as ketchup, sauces, dried vegetables, frozen vegetables, pre-cooked vegetables, preserved vegetables), snack articles (such as baked or deep-fried potato chips or potato dough products, extrudates based on maize or peanuts), fat- and oil-based products or emulsions thereof (such as mayonnaise, remoulade, dressings), other ready meals and soups (such as dry soups, instant soups, pre-cooked soups), spices, seasoning mixtures and, in particular, sprinkled seasonings: (English: seasonings), which are used, for example, in the snack sector. The inventive aroma mixture can in particular be used in sports drinks, including in particular those sports drinks which serve in athlete regeneration after intensive sportive activity or which increase performance.

If an aroma mixture is used, it is usually added in quantities of about 0.01 to 5, preferably about 0,1 to 3 and in particular about 1 to 2% by weight.

1. Capsules

If the products are food supplements, they are generally used without any further additives, with the exception of pure packaging materials.

Thus, according to a preferred embodiment of the invention, the aroma preparation can be encapsulated and then also added to foods as capsules. Capsules are spherical aggregates containing at least one solid or liquid core that is enclosed by at least one continuous shell.

Macrocapsules or microcapsules are a preferred form of application here. Macrocapsules preferably consist of gelatin or spray-dried products based on polysaccharides or dextrins. These generally have particle diameters of 0.5 to 1.5 cm.

However, the aroma mixture can also be encapsulated by coating materials and is thus available as macrocapsules with diameters of approximately 0.1 to approximately 5 mm or microcapsules with diameters of approximately 0.0001 to approximately 0.1 mm.

The terms “microcapsule” or “nanocapsule” are understood by those skilled in the art as spherical aggregates with a diameter in the range from about 0.0001 to about 5, and preferably 0.005 to 0.5 mm, containing at least one solid or liquid core which is enclosed by at least one continuous shell. More precisely, these are finely dispersed liquid or solid phases coated with film-forming polymers, in the production of which the polymers are deposited onto the material to be encapsulated after emulsification and coacervation or interfacial polymerization. According to another process, molten waxes are taken up in a matrix (“microsponge”), which, as microparticles, can be additionally coated with film-forming polymers. According to a third process, particles are alternatingly coated with polyelectrolytes of different charges (“layer-by-layer” process). The microscopically small capsules can be dried in the same way as powders. Besides single-core microcapsules, multi-core aggregates, also known as microspheres, are also known, which contain two or more cores distributed in the continuous coating material. In addition, single- or multi-core microcapsules can be enclosed by an additional second, third, etc. shell. The shell can consist of natural, semi-synthetic or synthetic materials. Natural coating materials include gum arabic, agar-agar, agarose, maltodextrins, alginic acid or salts thereof, e.g. sodium or calcium alginate, fats and fatty acids, cetyl alcohol, collagen, chitosan, lecithin, gelatin, albumin, shellac, polysaccharides such as starch or dextran, polypeptides, protein hydrolysates, sucrose and waxes. Semi-synthetic coating materials are inter alia chemically modified celluloses, in particular cellulose esters and ethers, e.g. cellulose acetate, ethyl cellulose, hydroxypropyl cellulose, hydroxypropyl methyl cellulose and carboxymethyl cellulose, as well as starch derivatives, in particular starch ethers and esters. Synthetic coating materials are, for example, polymers such as polyacrylates, polyamides, polyvinyl alcohol or polyvinylpyrrolidone.

Thereby, suitable coating materials are, for example, starches, including their degradation products as well as chemically or physically produced derivatives (particularly dextrins and maltodextrins), gelatin, gum arabic, agar-agar, ghatti gum, gellan gum, modified and non-modified celluloses, pullulan, curdlan, carrageenans, alginic acid, alginates, pectin, inulin, xanthan gum and mixtures of two or more of these substances.

The solid encapsulating material is preferably a gelatin (in particular porcine, bovine, poultry and/or fish gelatin), which, preferably has a swelling factor of more than or equal to 20, preferably more than or equal to 24. Among these substances, gelatin is particularly preferred, as it is readily available and can be purchased with different swelling factors.

Maltodextrins are also preferred (particularly based on cereals, especially maize, wheat, tapioca or potatoes), which preferably have DE values in the range of 10 to 20. Further preferred are celluloses (for example, cellulose ether), alginates (for example, sodium alginate), carrageenan (for example, beta-, jota-, lambda- and/or kappa-carrageenan), gum arabic, curdlan and/or agar agar.

Also preferred are alginate capsules as described in detail in the following documents: EP 0389700 A1, U.S. Pat. Nos. 4,251,195, 6,214,376, WO 2003 055587 or WO 2004 050069 A1.

In another preferred embodiment, the shell of the capsules consists of melamine-formaldehyde resins or coacervation products of cationic monomers or biopolymers (such as chitosan) and anionic monomers, such as (meth)acrylates or alginates.

Encapsulation Process

Generally, the capsules are finely dispersed liquid or solid phases coated with film-forming polymers, in the production of which the polymers are deposited onto the material to be coated after emulsification and coacervation or interfacial polymerisation. In another process, molten waxes are taken up into a matrix (“microsponge”), which, as microparticles, can be additionally coated with film-forming polymers. According to a third process, particles are alternatingly coated with polyelectrolytes of different charges (“layer-by-layer” process). The microscopically small capsules can be dried in the same way as powders. Besides single-core microcapsules, multi-core aggregates, also known as microspheres, are also known, which contain two or more cores distributed in the continuous coating material. In addition, single- or multi-core microcapsules can be enclosed by an additional second, third, etc. shell. The shell can consist of natural, semi-synthetic or synthetic materials. Natural coating materials include gum arabic, agar-agar, agarose, maltodextrins, alginic acid or salts thereof, e.g. sodium or calcium alginate, fats and fatty acids, cetyl alcohol, collagen, chitosan, lecithin, gelatin, albumin, shellac, polysaccharides such as starch or dextran, polypeptides, protein hydrolysates, sucrose and waxes. Semi-synthetic coating materials are inter alia chemically modified celluloses, in particular cellulose esters and ethers, e.g. cellulose acetate, ethyl cellulose, hydroxypropyl cellulose, hydroxypropyl methylcellulose and carboxymethyl cellulose, as well as starch derivatives, in particular starch ethers and esters. Synthetic coating materials are polymers such as polyacrylates, polyamides, polyvinyl alcohol or polyvinylpyrrolidone.

Examples of state of the art microcapsules are the following commercial products (the coating material is given in brackets): Hallcrest Microcapsules (gelatin, gum arabic), Coletica Thalaspheres (maritime collagen), Lipotec Millicapsules (alginic acid, agar-agar), Induchem Unispheres (lactose, microcrystalline cellulose, hydroxypropyl methylcellulose); Unicerin C30 (lactose, microcrystalline cellulose, hydroxypropyl methylcellulose), Kobo Glycospheres (modified starch, fatty acid esters, phospholipids), Softspheres (modified agar-agar) and Kuhs Probiol Nanospheres (phospholipids) as well as Primaspheres and Primasponges (chitosan, alginates) and Primasys (phospholipids).

Chitosan microcapsules and processes for their production are well known from the state of the art [WO 01/01926, WO 01/01927, WO 01/01928, WO 01/01929]. Microcapsules with mean diameters in the range from 0.0001 to 5, preferably 0.001 to 0.5 and particularly 0.005 to 0.1 mm, consisting of a coating membrane and a matrix containing the active substances, can be obtained, for example, by

(a) preparing a matrix from gel forming agents, cationic polymers and active substances,

(b) optionally, dispersing the matrix in an oil phase,

(c) treating the dispersed matrix with aqueous solutions of anionic polymers, optionally thereby removing the oil phase.

Thereby, steps (a) and (c) are exchangeable insofar as anionic polymers are used instead of cationic polymers in step (a) and vice versa.

The capsules can also be produced by alternatingly coating the active substance with layers of differently charged polyelectrolytes (layer-by-layer technology). In this context, reference is made to the European Patent EP 1064088 B1 (Max-Planck Society).

Pharmaceutical Preparations

Another object of the invention concerns pharmaceutical preparations for the protection of the human or animal body containing an inventive aroma mixture.

As far as pharmaceutical preparations are concerned, the same considerations apply to them as have already been explained above for food supplements. The fact is that these preparations are actually to be regarded as so-called “neutraceuticals” or “cosmeceuticals” and are therefore in the overlapping area of pharmaceuticals, foods and cosmetics. If the preparations are taken orally, in this context one speaks also of a “beauty from inside” effect, because thereby as skin aging should be prevented.

Cosmetic Agents

A further object of the invention concerns cosmetic agents containing an inventive aroma mixture.

The inventive cosmetic agents may contain further typical auxiliaries and additives, such as mild surfactants, oil bodies, emulsifiers, pearlescent waxes, consistency enhancers, thickeners, superfatting agents, stabilizers, polymers, silicone compounds, fats, waxes, lecithins, phospholipids, UV light protection factors, humectants, biogenic active ingredients, antioxidants, deodorants, antiperspirants, anti-dandruff agents, film formers, swelling agents, insect repellents, self-tanning agents, tyrosine inhibitors (depigmentation agents), hydrotropes, solubilizers, preservatives, perfume oils, dyes and the like. Since many pharmaceutical preparations contain similar ingredients, the following examples also apply here.

1. Surfactants

As surface-active substances, anionic, nonionic, cationic and/or amphoteric or zwitterionic surfactants may be contained, the proportion of which in the agents is usually about 1 to 70, preferably 5 to 50 and in particular 10 to 30 wt. %. Typical examples of anionic surfactants are soaps, alkylbenzene sulfonates, alkane sulfonates, olefin sulfonates, alkyl ether sulfonates, glyceryl ether sulfonates, α-methyl ester sulfonates, sulfofatty acids, alkyl sulphates, alkyl ether sulphates, glyceryl ether sulphates, fatty acid ether sulfates, hydroxy mixed ether sulphates, monoglyceride (ether) sulphates, fatty acid amide (ether) sulfates, mono and dialkylsulfosuccinates, mono- and dialkyl sulfosuccinamates, sulfotriglycerides, amide soaps, ether carboxylic acids and salts thereof, fatty acid isothionates, fatty acid sarcosinates, fatty acid taurides, N-acylamino acids, such as acyl lactylates, acyl tartrates, acyl glutamates and acyl aspartates, alkyl oligoglucoside sulfates, protein fatty acid condensates (in particular plant products based on wheat) and alkyl (ether) phosphates. If the anionic surfactants contain polyglycol ether chains, these may have a conventional, but preferably narrowed homolog distribution. Typical examples of non-ionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers or mixed formals, optionally partially oxidized alk(en)yl oligoglycosides or glucoronic acid derivatives, fatty acid N-alkyl glucamides, protein hydrolysates (in particular plant products based on wheat), polyol fatty acid esters, sucrose esters, sorbitan esters, polysorbates and amine oxides. If the non-ionic surfactants contain polyglycol ether chains, these may have a conventional, but preferably narrowed homolog distribution. Typical examples of cationic surfactants are quaternary ammonium compounds, such as dimethyl distearyl ammonium chloride, and esterquats, in particular quaternized fatty acid trialkanolamine ester salts. Typical examples of amphoteric or zwitterionic surfactants are alkyl betaines, alkyl amidobetaines, aminopropionates, aminoglycinates, imidazolinium betaines and sulfobetaines. The surfactants mentioned are exclusively known compounds. Typical examples of particularly suitable mild, i.e. particularly skin-friendly surfactants are fatty alcohol polyglycol ether sulfates, monoglyceride sulfates, mono- and/or dialkyl sulfosuccinates, fatty acid isethionates, fatty acid sarcosinates, fatty acid taurides, fatty acid glutamates, α-olefin sulfonates, ether carboxylic acids, alkyl oligoglucosides, fatty acid glucamides, alkyl amido betaines, amphoacetals and/or protein fatty acid condensates, the latter preferably based on wheat proteins.

2. Oil Bodies

Oil bodies can be, for example, Guerbet alcohols based on fatty alcohols having 6 to 18, preferably 8 to 10, carbon atoms, esters of linear C6-C22-fatty acids with linear or branched C6-C22-fatty alcohols, or esters of branched C6-C13-carboxylic acids with linear or branched C6-C22-fatty alcohols, such as myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of linear C6-C22-fatty acids with branched alcohols, in particular 2-ethylhexanol, esters of C18-C38-alkylhydroxy carboxylic acids with linear or branched C6-C22-fatty alcohols, in particular dioctyl malates, esters of linear and/or branched fatty acids with polyhydric alcohols (such as, for example, propylene glycol, dimerdiol or trimertriol) and/or Guerbet alcohols, triglycerides based on C6-C10-fatty acids, liquid mono-/di-/triglyceride mixtures based on C6-C18-fatty acids, esters of C6-C22-fatty alcohols and/or Guerbet alcohols with aromatic carboxylic acids, in particular benzoic acid, esters of C2-C12-dicarboxylic acids with linear or branched alcohols having 1 to 22 carbon atoms or polyols having 2 to 10 carbon atoms and 2 to 6 hydroxyl groups, vegetable oils, branched primary alcohols, substituted cyclohexanes, linear and branched C6-C22-fatty alcohol carbonates, such as, for example, Dicaprylyl Carbonate (Cetiol<®>CC), Guerbet carbonates based on fatty alcohols having 6 to 18, preferably from 8 to 10 C atoms, esters of benzoic acid with linear and/or branched C6-C22-alcohols (e.g. Finsolv® TN), linear or branched, symmetrical or asymmetrical dialkyl ethers having 6 to 22 carbon atoms per alkyl group, such as, for example, dicaprylyl ether (Cetiol<®>OE), ring opening products of epoxidized fatty acid esters with polyols, silicone oils (cyclomethicones, silicon-methicone types, etc.) and/or aliphatic or naphthenic hydrocarbons such as, for example, squalane, squalene or dialkylcyclohexanes.

3. Emulsifiers

As emulsifiers, for example, non-ionic surfactants from at least one of the following groups can be used:
Addition products of 2 to 30 mol of ethylene oxide and/or 0 to 5 mol of propylene oxide to linear fatty alcohols having 8 to 22 carbon atoms, to fatty acids having 12 to 22 carbon atoms, to alkylphenols having 8 to 15 carbon atoms in the alkyl group as well as to alkylamines having 8 to 22 carbon atoms in the alkyl radical;
Alkyl and/or alkenyl oligoglycosides having 8 to 22 carbon atoms in the alk(en)yl radical and ethoxylated analogues thereof;
Addition products of 1 to 15 mol ethylene oxide to castor oil and/or hardened castor oil;
Addition products of 15 to 60 mol ethylene oxide to castor oil and/or hardened castor oil;
Partial esters of glycerol and/or sorbitan with unsaturated, linear or saturated, branched fatty acids having 12 to 22 carbon atoms and/or hydroxycarboxylic acids having 3 to 18 carbon atoms and adducts thereof with 1 to 30 mol of ethylene oxide;
Partial esters of polyglycerol (average degree of self-condensation 2 to 8), polyethylene glycol (molecular weight 400 to 5000), trimethylolpropane, pentaerythritol, sugar alcohols (e.g. sorbitol), alkyl glucosides (e.g. methyl glucoside, butyl glucoside, lauryl glucoside) and polyglucosides (e.g. cellulose) with saturated and/or unsaturated, linear or branched fatty acids having 12 to 22 carbon atoms and/or hydroxycarboxylic acids having 3 to 18 carbon atoms and adducts thereof with 1 to 30 mol of ethylene oxide;
Mixed ester of pentaerythritol, fatty acids, citric acid and fatty alcohol and/or mixed ester of fatty acids having 6 to 22 carbon atoms, methylglucose and polyols, preferably glycerol or polyglycerol.
Mono-, di- and trialkyl phosphates as well as mono-, di- and/or tri-PEG-alkyl phosphates and salts thereof;
Wool wax alcohols;
Polysiloxane-polyalkyl-polyether copolymers or corresponding derivatives;
Block copolymers e.g. polyethylene glycol-30 dipolyhydroxy stearate;
Polymer emulsifiers, e.g. Pemulen type (TR-1, TR-2) from Goodrich or Cosmedia<®>SP from Cognis;
Polyalkylene glycols as well as
Glycerol carbonate.

In the following, particularly suitable emulsifiers are described in further detail:

(i) Alkoxylates. The addition products of ethylene oxide and/or propylene oxide to fatty alcohols, fatty acids, alkylphenols or castor oil are known, commercially available products. These are homolog mixtures whose average degree of alkoxylation corresponds to the ratio between the amount of substance of ethylene oxide and/or propylene oxide and the substrate with which the addition reaction is carried out. C12/18 fatty acid mono- and diesters of addition products of ethylene oxide to glycerol are known as re-fattening agents for cosmetic preparations.

(ii) Alkyl and/or alkenyl oligoglycosides. Alkyl and/or alkenyl oligoglycosides, their preparation and use are known from the state of the art. Their production is carried out in particular by reacting glucose or oligosaccharides with primary alcohols having 8 to 18 carbon atoms. With respect to the glycoside radical, both monoglycosides, in which a cyclic sugar radical is glycosidically bound to the fatty alcohol, and oligomeric glycosides having a degree of oligomerization up to preferably about 8 are suitable. Here, the degree of oligomerisation is a statistical mean value which is based on a homolog distribution common for such technical products.

(iii) Partial glycerides. Typical examples of suitable partial glycerides are hydroxystearic acid monoglyceride, hydroxystearic acid diglyceride, isostearic acid monoglyceride, isostearic acid diglyceride, oleic acid monoglyceride, oleic acid diglyceride, ricinoleic acid monoglyceride, ricinoleic acid diglyceride, linoleic acid monoglyceride, linoleic acid diglyceride, linolenic acid monoglyceride, linolenic acid diglyceride, erucic acid monoglyceride, erucic acid diglyceride, tartaric acid monoglyceride, tartaric acid diglyceride, citric acid monoglyceride, citron diglyceride, malic acid monoglyceride, malic acid diglyceride and technical mixtures thereof, which may also secondarily comprise small amounts of triglyceride from the production process. Addition products of 1 to 30, preferably 5 to 10 mol ethylene oxide to the partial glycerides mentioned are also suitable.

(iv) Sorbitan esters. The sorbitan esters used are sorbitan monoisostearate, sorbitan sesquiisostearate, sorbitan diisostearate, sorbitan triisostearate, sorbitan monooleate, sorbitan sesquioleate and sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesquihydroxystearate, sorbitan dihydroxystearate, sorbitan trihydroxystearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate and technical mixtures thereof. Also suitable are addition products of 1 to 30, preferably 5 to 10 mol ethylene oxide to the mentioned sorbitan esters.

(v) Polyglycerol esters. Typical examples of suitable polyglycerol esters are polyglyceryl-2 dipolyhydroxystearate (Dehymuls® PGPH), polyglycerol-3 diisostearate (Lameform® TGI), polyglyceryl-4 isostearate (Isolan® GI 34), polyglyceryl-3 oleate, diisostearoyl polyglyceryl-3 diisostearate (Isolan® PDI), polyglyceryl-3 methylglucose distearate (Tego Care® 450), polyglyceryl-3 beeswax (Cera Bellina®), polyglyceryl-4 caprate (polyglycerol caprate T2010/90), polyglyceryl-3 cetyl ether (Chimexane® NL), polyglyceryl-3 distearate (Cremophor® GS 32) and polyglyceryl polyricinoleate (Admul® WOL 1403) polyglyceryl dimerate isostearate, as well as mixtures thereof. Examples of further suitable polyol esters are the mono-, di- and triesters of trimethylol propane or pentaerythritol, optionally reacted with 1 to 30 mol ethylene oxide, with lauric acid, coconut fatty acid, tallow fatty acid, palmitic acid, stearic acid, oleic acid, behenic acid and the like.

(vi) Anionic emulsifiers. Typical anionic emulsifiers are aliphatic fatty acids with 12 to 22 carbon atoms, such as for example palmitic acid, stearic acid or behenic acid, as well as dicarboxylic acids with 12 to 22 carbon atoms, such as, for example, azelaic acid or sebacic acid.

(vii) Amphoteric and cationic emulsifiers. Moreover, zwitterionic surfactants can be used as emulsifiers. Zwitterionic surfactants are surface-active compounds which contain at least one quaternary ammonium group and at least one carboxylate and one sulfonate group in the molecule. Particularly suitable zwitterionic surfactants are the so-called betaines, such as the N-alkyl-N,N-dimethyl ammonium glycinates, for example the cocoalkyl dimethyl ammonium glycinate, N-acylaminopropyl-N,N-dimethyl ammonium glycinates, for example the cocoacylaminopropyl dimethyl ammonium glycinate, and 2-alkyl-3-carboxylmethyl-3-hydroxyethyl imidazolines each having 8 to 18 carbon atoms in the alkyl or acyl group, as well as the cocoacylaminoethyl hydroxyethyl carboxymethyl glycinate. The fatty acid amide derivative known under the CFT name of Cocamidopropyl Betaine is particularly preferred. Ampholytic surfactants are also suitable emulsifiers. Ampholytic surfactants are surface-active compounds which, in addition to a C8/18-alkyl or acyl, contain at least one free amino group and at least one —COOH— or —SO3H-group in the molecule and which are capable of forming inner salts. Examples of suitable ampholytic surfactants are N-alkyl glycines, N-alkyl propionic acids, N-alkylaminobutyric acids, N-alkyliminodipropionic acids, N-hydroxyethyl-N-alkylamidopropyl glycines, N-alkyl taurines, N-alkyl sarcosines, 2-alkylaminopropionic acids and alkylaminoacetic acids each having about 8 to 18 carbon atoms in the alkyl group. Particularly preferred ampholytic surfactants are N-cocoalkylaminopropionate, cocoacylaminoethyl aminopropionate and C12/18-acylsarcosine. Finally, cationic surfactants can also be considered as emulsifiers, whereby those of the esterquat type, preferably methylquaternized difatty acid triethanolamine ester salts, are particularly preferred.

4. Fats and Waxes

Typical examples of fats are glycerides, i.e. solid or liquid plant or animal products, which essentially consist of mixed glycerol esters of higher fatty acids, and as natural waxes, such as candelilla wax, carnauba wax, Japan wax, espartograss wax, cork wax, guaruma wax, rice germ oil wax, sugar cane wax, ouricury wax, montan wax, beeswax, shellac wax, spermaceti, lanolin (wool wax), uropygial fat, ceresine, ozokerite (earth wax), petrolatum, paraffin wax, microwaxes; chemically modified waxes (hard waxes), such as montan ester waxes, Sasol waxes, hydrogenated jojoba waxes as well as synthetic waxes such as polyalkylene waxes and polyethylene glycol waxes are possible. In addition to fats, fat-like substances such as lecithins and phospholipids can also be used as additives. Lecithins are those glycero-phospholipids which are formed from fatty acids, glycerol, phosphoric acid and choline by esterification. Lecithins are therefore also often referred to as phosphatidylcholine (PC) in the scientific community. Examples of natural lecithins are cephalins, also known as phosphatidic acids, which are derivatives of 1,2-diacyl-sn-glycerol-3-phosphoric acids. By contrast, phospholipids are usually understood to be mono- and preferably diesters of phosphoric acid with glycerol (glycerol phosphates), which are generally classified as fats. Besides these, sphingosines and sphingolipids are also possible.

5. Pearlescent Waxes

As pearlescent waxes, the following are possible, for example: Alkylene glycol esters, especially ethylene glycol distearate; fatty acid alkanolamides, especially coco fatty acid diethanolamide; partial glycerides, especially stearic acid monoglyceride; esters of polyvalent, optionally hydroxy-substituted carboxylic acids with fatty alcohols having 6 to 22 carbon atoms, especially long-chain esters of tartaric acid; fatty substances, such as fatty alcohols, fatty ketones, fatty aldehydes, fatty ethers and fatty carbonates, which in total have at least 24 carbon atoms, especially laurone and distearyl ether; fatty acids such as stearic acid, hydroxystearic acid or behenic acid, ring opening products of olefin epoxides having 12 to 22 carbon atoms with fatty alcohols having 12 to 22 carbon atoms and/or polyols having 2 to 15 carbon atoms and 2 to 10 hydroxyl groups and mixtures thereof.

6. Cooling Substances

Cooling substances are compounds that create a feeling of cold on the skin. As a rule, these are menthol compounds which—in addition to the basic menthol structure itself, menthol—are selected from the group consisting of menthol methyl ether, menthone glyceryl acetal (FEMA GRAS<1>3807), menthone glyceryl ketal (FEMA GRAS 3808), menthyl lactate (FEMA GRAS 3748), menthol ethylene glycol carbonate (FEMA GRAS 3805), menthol propylene glycol carbonate (FEMA GRAS 3806), menthyl N-ethyloxamate, monomethyl succinate (FEMA GRAS 3810), monomenthyl glutamate (FEMA GRAS 4006), menthoxy-1,2-propanediol (FEMA GRAS 3784), menthoxy-2-methyl-1,2-propanediol (FEMA GRAS 3849) as well as the menthane-carboxylic acid esters and amides WS-3, WS-4, WS-5, WS-12, WS-14 and WS-30 and mixtures thereof.

<1>FEMA stands for “Flavor and Extracts Manufacturers Association” and GRAS is defined as “Generally Regarded As Safe”. A FEMA GRAS designation means that the substance thus labelled is tested according to the standard method and considered toxicologically harmless.

A first important representative of these substances is the monomenthyl succinate (FEMA GRAS 3810). Both succinate and analog monomenthyl glutarate (FEMA GRAS 4006) are important representatives of monomenthyl esters based on di- and polycarboxylic acids:

Examples of applications of these substances can be found in the publications WO 2003 043431 (Unilever) or EP 1332772 A1 (IFF).

The next important group of menthol compounds preferred in the sense of the invention comprises carbonate esters of menthol and polyols, such as glycols, glycerol or carbohydrates, such as menthol ethylene glycol carbonates (FEMA GRAS 3805=Frescolat< [symbol] >MGC), menthol propylene glycol carbonates (FEMA GRAS 3784=Frescolat< [symbol] >MPC), menthol 2-methyl-1,2-propanediol carbonates (FEMA GRAS 3849) or the corresponding sugar derivatives. The menthol compounds menthyl lactate (FEMA GRAS 3748=Frescolat< [symbol] >ML) and in particular menthone glyceryl acetal (FEMA GRAS 3807) or menthone glyceryl ketal (FEMA GRAS 3808), which is marketed under the name Frescolat< [symbol] >MGA, are also preferred. Among these substances, menthone glyceryl acetal/ketal as well as menthyl lactate and menthol ethylene glycol carbonate and menthol propylene glycol carbonate, respectively, which the applicant markets under the names Frescolat< [symbol] >MGA, Frescolat< [symbol] >ML, Frecolat< [symbol] >MGC and Frescolat< [symbol] >MPC, have proved to be particularly advantageous.

In the 70s of the last century, menthol compounds were developed for the first time which have a C—C bond in the 3-position and of which a number of representatives can also be used. These substances are generally referred to as WS types. The basic structure is a menthol derivative in which the hydroxyl group is replaced by a carboxyl group (WS-1). All other WS types are derived from this structure, such as the preferred species WS-3, WS-4, WS-5, WS-12, WS-14 and WS-30.

7. Consistency Enhancers and Thickeners

As consistency enhancers primarily fatty alcohols or hydroxy fatty alcohols with 12 to 22 and preferably 16 to 18 carbon atoms and besides these also partial glycerides, fatty acids or hydroxy fatty acids are considered. A combination of these substances with alkyl oligoglucosides and/or fatty acid N-methyl glucamides of the same chain length and/or polyglycerol poly-12-hydroxystearates is preferred. Suitable thickeners are for example aerosil types (hydrophilic silicas), polysaccharides, in particular xanthan gum, guar guar, agar-agar, alginates and tyloses, carboxymethyl cellulose and hydroxyethyl- and hydroxypropylcellulose, as well as higher molecular weight polyethylene glycol mono- and diesters of fatty acids, polyacrylates, (e.g. Carbopole [symbol] and Pemulen types from Goodrich; Synthalene [symbol] from Sigma; Keltrol types from Kelco; Sepigel types from Seppic; Salcare types from Allied Colloids), polyacrylamides, polymers, polyvinyl alcohol and polyvinyl pyrrolidone. Bentonites such as Bentone< [symbol] >Gel VS-5PC (Rheox), a mixture of cyclopentasiloxane, disteardimonium hectorite and propylene carbonate, have also proven to be particularly effective. Further considered are surfactants, such as ethoxylated fatty acid glycerides, esters of fatty acids with polyols such as pentaerythritol or trimethylolpropane, fatty alcohol ethoxylates with narrowed homolog distribution or alkyl oligoglucosides as well as electrolytes such as sodium chloride and ammonium chloride.

8. Superfatting Agents and Stabilizers

As superfatting agents substances such as lanolin and lecithin as well as polyethoxylated or acylated lanolin and lecithin derivatives, polyol fatty acid esters, monoglycerides and fatty acid alkanolamides can be used, the latter also serving as foam stabilizers.

Metal salts of fatty acids such as, for example, magnesium, aluminium and/or zinc stearate or ricinoleate, respectively, can be used as stabilizers.

9. Polymers

Suitable cationic polymers are, for example, cationic cellulose derivatives such as, for example, quaternized hydroxy ethylcellulose, which is available under the name Polymer JR 400 [symbol] from Amerchol, cationic starch, copolymers of diallyl ammonium salts and acrylamides, quaternized vinyl pyrrolidone/vinyl imidazole polymers such as, for example, Luviquat [symbol] (BASF), condensation products of polyglycols and amines, quaternized collagen polypeptides, such as, for example, Lauryldimonium Hydroxypropyl Hydrolyzed Collagen (Lamequat [symbol] L/Grünau), quaternized wheat polypeptides, polyethyleneimine, cationic silicone polymers, such as, for example, amodimethicones, copolymers of adipic acid and dimethylaminohydropyl diethylenetriamine (Cartaretine [symbol] /Sandoz), copolymers of acrylic acid with dimethyl diallyl ammonium chloride (Merquat [symbol] 550/Chemviron), polyaminopolyamides and crosslinked water-soluble polymers thereof, cationic chitin derivatives such as, for example, quaternized chitosan, optionally microcrystalline distributed, condensation products of dihalogenalkyls, such as, for example, dibromobutane with bisdialkylamines, such as bis-dimethylamino-1,3-propane, cationic guar gum, such as Jaguar [symbol] CBS, Jaguar [symbol] C-17, Jaguar [symbol] C-16 from Celanese, quaternized ammonium salt polymers, such as Mirapol [symbol] A-15, Mirapol [symbol] AD-1, Mirapol [symbol] AZ-1 from Miranol.

Considered as anionic, zwitterionic, amphoteric and non-ionic polymers are for example vinyl acetate/crotonic acid copolymers, vinyl pyrrolidone/vinyl acrylate copolymers, vinyl acetate/butyl maleate/isobornyl acrylate copolymers, methyl vinyl ether/maleic anhydride copolymers and esters thereof, uncrosslinked and polyol-crosslinked polyacrylic acids, acrylamidopropyl trimethylammonium chloride/acrylate copolymers, octylacrylamide/methyl methacrylate/tert.-butylaminoethylmethacrylate/2-hydroxypropylmethacrylate copolymers, polyvinylpyrrolidone, vinylpyrrolidone/vinyl acetate copolymers, vinylpyrrolidone/dimethylaminoethyl methacrylate/vinylcaprolactam terpolymers and optionally derivatized cellulose ethers and silicones.

10. Silicone Compounds

Suitable silicone compounds are, for example, dimethylpolysiloxanes, methylphenyl polysiloxanes, cyclic silicones as well as amino-, fatty acid-, alcohol-, polyether-, epoxy-, fluorine-, glycoside- and/or alkyl-modified silicone compounds, which may be both liquid and resin-like at room temperature. Further suitable are simethicones, which are mixtures of dimethicones with an average chain length of 200 to 300 dimethylsiloxane units and hydrogenated silicates.

11. UV Light Protection Factors

Under UV light protection factors, for example, organic substances (light protection filters) which are liquid or crystalline at room temperature are to be understood, which are able to absorb ultraviolet rays and release the absorbed energy in the form of longer-waved radiation, e.g. heat. Usually, UV light protection factors are added in amounts of 0.1 to 5 and preferably 0.2 to 1 wt. % by UVB filters can be oil soluble or water soluble. As oil-soluble substances to be mentioned are for example:
3-benzylidenecamphor or 3-benzylidennorcamphor and its derivatives, respectively, e.g. 3-(4-methylbenzylidene)camphor;
4-aminobenzoic acid derivatives, preferably 4-(dimethylamino)benzoic acid-2-ethylhexyl ester, 4-(dimethylamino)benzoic acid-2-octyl ester and 4-(dimethylamino)benzoic acid amyl ester;
esters of cinnamic acid, preferably 4-methoxy cinnamic acid-2-ethylhexyl ester, 4-methoxy cinnamic acid-propyl ester, 4-methoxy cinnamic acid-isoamyl ester 2-cyano-3,3-phenyl cinnamic acid-2-ethylhexyl ester (octocrylenes);
esters of salicylic acid, preferably salicylic acid 2-ethylhexyl ester, salicylic acid 4-isopropylbenzyl ester, salicylic acid homomenthyl ester;
benzophenone derivatives, preferably 2-hydroxy-4-methoxybenzophenone, 2-hydroxy-4-methoxy-4′-methylbenzophenone, 2,2′-dihydroxy-4-methoxybenzophenone;
esters of benzalmalonic acid, preferably 4-methoxybenzmalonic acid di-2-ethylhexyl esters;
triazine derivatives such as 2,4,6-trianilino-(p-carbo-2′-ethyl-1′-hexyloxy)-1,3,5-triazine and octyl triazone or dioctyl butamido triazone (Uvasorb [symbol] HEB);
propane-1,3-diones, such as, for example, 1-(4-tert.-butylphenyl)-3-(4′-methoxyphenyl) propane-1,3-dione;
ketotricyclo(5.2.1.0)decane derivatives.

Considered as water-soluble substances are:
2-phenylbenzimidazole-5-sulfonic acid and alkali metal-, alkaline earth metal-, ammonium-, alkylammonium-, alkanolammonium- and glucammonium salts thereof;
1H-benzimidazole-4,6-disulfonic acid, 2,2′-(1,4-phenylene)bis-, disodium salt (Neo Heliopan< [symbol] >AP)
sufonic acid derivatives of benzophenones, preferably 2-hydroxy-4-methoxy benzophenone-5-sulphonic acid and salts thereof;
sulfonic acid derivatives of 3-benzylidenecamphor such as 4-(2-oxo-3-bornylidenemethyl benzenesulfonic acid and 2-methyl-5-(2-oxo-3-bornylidene)sulfonic acid and salts thereof.

Considered as typical UV-A filters are benzoylmethane derivatives, such as 1-(4′-tert.-butylphenyl)-3-(4′-methoxyphenyl)propane-1,3-dione, 4-tert.-butyl-4′-methoxy dibenzoylmethane (Parsol [symbol] 1789), 2-(4-diethylamino-2-hydroxybenzoyl)-benzoic acid hexyl ester (Uvinul [symbol] A Plus), 1-phenyl-3-(4′-isopropylphenyl)-propane-1,3-dione and enamine compounds. The UV-A and UV-B filters can of course also be used in mixtures. Particularly favorable combinations consist of the derivatives of benzoylmethane, e.g. 4-tert.-butyl-4′-methoxydibenzoylmethane (Parsol [symbol] 1789) and 2-cyano-3,3-phenylcinnamic acid 2-ethylhexylester (Octocrylene) in combination with esters of cinnamic acid, preferably 4-methoxycinnamic acid 2-ethylhexylester and/or 4-methoxycinnamic acid propylester and/or 4-methoxycinnamic acid isoamylester. Such combinations are advantageously combined with water-soluble filters such as, for example, 2-phenylbenzimidazole-5-sulfonic acid and alkali, alkaline earth, ammonium, alkylammonium, alkanolammonium and glucammonium salts thereof.

In addition to the soluble substances mentioned above, insoluble light protection pigments, i.e. finely dispersed metal oxides or salts, can also be considered for this purpose. Examples of suitable metal oxides are in particular zinc oxide and titanium dioxide and also oxides of iron, zirconium, silicon, manganese, aluminium and cerium as well as mixtures thereof. Silicates (talc), barium sulfate or zinc stearate can be used as salts. Oxides and salts are used in the form of pigments for skin caring and skin protecting emulsions and decorative cosmetics. The mean diameter of the particles should be less than 100 nm, preferably between 5 and 50 nm and in particular between 15 and 30 nm. They may have a spherical shape, however, particles with an ellipsoidal shape or a shape differing from the spherical shape may be used as well. The pigments can also be surface-treated, i.e. hydrophilized or hydrophobized. Typical examples are coated titanium dioxides, such as e.g. titanium dioxide T 805 (Degussa) or Eusolex< [symbol] >T2000, Eusolex< [symbol] >T, Eusolex< [symbol] >T-ECO, Eusolex< [symbol] >T-S, Eusolex< [symbol] >T-Aqua, Eusolex< [symbol] >T-45D (all Merck), Uvinul TiO2 (BASF). Silicones and in particular trialkoxyoctylsilanes or simethicones can be considered as hydrophobic coating agents. So-called micro- or nanopigments are preferably used in suns protection agents. Micronized zinc oxide such as, for example, Z-COTE< [symbol] > or Z-COTE HP1< [symbol] > is preferably used.

12. Humectant Agent

Humectant agents serve to further optimize the sensory properties of the composition and the regulation of the skin. At the same time, the cold stability of the preparations in conformity with the invention is increased, particularly in the case of emulsions. The humectants are usually contained in an amount of 0.1 to 15 wt. %, preferably 1 to 10 wt. %, and more particular 5 to 10 wt. %.

Suitable according to the present invention are among others amino acids, pyrrolidonecarboxylic acid, lactic acid and salts thereof, lactitol, urea and urea derivatives, uric acid, glucosamine, creatinine, collagen cleavage products, chitosan or chitosan salts/derivatives, and in particular polyols and polyol derivatives (e.g. glycerol, diclycerol, triglycerol, ethylene glycol, propylene glycol, butylene glycol, erythritol, 1,2,6-hexanetriol, polyethylene glycols such as PEG-4, PEG-6, PEG-7, PEG-8, PEG-9, PEG-10, PEG-12, PEG-14, PEG-16, PEG-18, PEG-20), sugar and sugar derivatives (i.a. fructose, glucose, maltose, maltitol, mannite, inositol, sorbitol, sorbitylsilanediol, sucrose, trehalose, xylose, xylitol, glucuronic acid and salts thereof), ethoxylated sorbitol (Sorbeth-6, Sorbeth-20, Sorbeth-30, Sorbeth-40), honey and hardened honey, hardened starch hydrolysates as well as mixtures of hardened wheat protein and PEG-20 acetate copolymer. According to the invention, glycerine, diglycerine, triglycerine and butylene glycol are preferably suitable as humectants.

13. Biogenic Active Ingredients and Antioxidants

Biogenic active ingredients are understood to be for example tocopherol, tocopherol acetate, tocopherol palmitate, ascorbic acid, (deoxy)ribonucleic acid and fragmentation products thereof, β-glucans, retinol, bisabolol, allantoin, phytantriol, panthenol, AHA acids, amino acids, ceramides, pseudoceramides, essential oils, plant extracts, such as e.g. prune extract, Bambara nut extract and vitamin complexes.

Antioxidants interrupt the photochemical reaction chain that is triggered when UV radiation penetrates the skin. Typical examples thereof are amino acids (e.g. glycine, histidine, tyrosine, tryptophan) and derivatives thereof, imidazoles (e.g. urocanic acid) and derivatives thereof, peptides such as D,L-carnosine, D-carnosine, L-carnosine and derivatives thereof (e.g. anserine), carotenoids, carotenes (e.g. α-carotene, β-carotene, lycopene) and derivatives thereof, chlorogenic acid and derivatives thereof, lipoic acid and derivatives thereof (e.g. dihydroliponic acid), aurothioglucose, propylthiouracil and other thiols (e.g. thioredoxin, glutathione, cysteine, cystine, cysteamine and glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) and sulfoximine compounds (e.g. buthionine sulfoximines, homocysteine sulfoximine, butionine sulfones, penta-, hexa-, heptathionine sulfoximine) in very low tolerated doses (e.g. pmol to μmol/kg), furthermore (metal) chelators (e.g. α-hydroxy fatty acids, palmitic acid, phytic acid, lactoferrin), α-hydroxy acids (e.g. citric acid, lactic acid, malic acid), humic acid, bile acids, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, ubiquinone and ubiquinol and its derivatives, vitamin C and derivatives (e.g. ascorbyl palmitate, Mg-ascorbyl phosphate, ascorbylacetate), tocopherols and derivatives (e.g. vitamin E acetate), vitamin A and derivatives (vitamin A palmitate) as well as coniferyl benzoate from benzoic resin, rutinic acid and derivatives thereof, α-glycosyl rutin, ferulic acid, furfurylidene glucitol, carnosine, butyl hydroxy toluene, butylhydroxyanisole, nordihydroguaiaretic resin acid, nordihydroguajaretic acid, trihydroxybutyrophenone, uric acid and derivatives thereof, mannose and derivatives thereof, superoxide dismutase, zinc and derivatives thereof (for example ZnO, ZnSO4) selenium and its derivatives (e.g. selenium-methionine), stilbenes and their derivatives (e.g. stilbene oxide, trans-stilbene oxide) and the inventive suitable derivates of the mentioned agents (salts, esters, ethers, sugars, nucleotides, nucleosides, peptides and lipids).

14. Hydrotropes

In addition, hydrotropes, for example, ethanol, isopropyl alcohol or polyols can be used to improve the flow behavior; these substances largely correspond to the carriers described above. Polyols, considered here, preferably have 2 to 15 carbon atoms and at least two hydroxyl groups. The polyols may also contain other functional groups, in particular amino groups, or may be modified with nitrogen. Typical examples are
Glycerol;
Alkylene glycols such as ethylene glycol, diethylene glycol, propylene glycol, butylene glycol, hexylene glycol and polyethylene glycols with an average molecular weight of 100 to 1,000 Dalton;
Technical oligoglycerine mixtures having a degree of self-condensation of 1.5 to 10, such as technical diglycerol mixtures with a diglycerol content of 40 to 50 wt. %;
Methyol compounds, in particular, trimethylol ethane, trimethylol propane, trimethylol butane, pentaerythritol and dipentaerythritol;
Low alkyl glucosides, in particular those with 1 to 8 carbons in the alkyl radical, such as methyl and butyl glucoside;
Sugar alcohols with 5 to 12 carbon atoms, such as sorbitol or mannitol,
Sugar with 5 to 12 carbon atoms, such as glucose or sucrose;
Amino sugars, such as glucamine;
Dialkoholamines, such as diethanolamine or 2-amino-1,3-propanediol.

15. Preservatives

Suitable preservatives are, for example, phenoxyethanol, formaldehyde solution, parabens, pentanediol or sorbic acid as well as the silver complexes known under the name Surfacine<®> and the other substance classes listed in Annex 6, Part A and B of the Cosmetics Directive.

16. Perfume Oils and Aromas

Perfume oils can be mixtures of natural and synthetic fragrances. Natural fragrances are extracts of blossoms (lily, lavender, rose, jasmine, neroli, ylang-ylang), stems and leaves (geranium, patchouli, petitgrain), fruits (aniseed, coriander, caraway, juniper), fruit peels (bergamot, lemon, orange), roots (macis, angelica, celery, cardamom, costus, iris, calmus), wood (pinewood, sandalwood, guaiacwood, cedarwood, rosewood), herbs and grasses (tarragon, lemon grass, sage, thyme), needles and branches (spruce, fir, pine, dwarf pine), resins and balsams (galbanum, elemi, benzoin, myrrh, olibanum, opoponax). Furthermore, considered are animal raw materials, such as cibet and castoreum. Typical synthetic fragrance compounds are products of the ester, ether, aldehyde, ketone, alcohol and hydrocarbon type. Fragrance compounds of the ester type are e.g. benzylacetate, phenoxyethyl isobutyrate, p-tert.-butylcyclohexylacetate, linalyl acetate, dimethyl benzyl carbinyl acetate, phenylethyl acetate, linalyl benzoate, benzylformate, ethylmethyl phenylglycinate, allyl cyclohexyl propionate, styrallyl propionate and benzylsalicylate. The ethers include benzylethyl ether, the aldehydes include e.g. the linear alkanals with 8 to 18 carbon atoms, citral, citronellal, citronellyloxyacetaldehyde, cyclamen aldehyde, hydroxy citronellal, lilyal and bourgeonal, the ketones include e.g. the ionones, α-isomethylionone and methylcedrylketone, the alcohols include A-nethol, citronellol, eugenol, isoeugenol, geraniol, linalool, phenylethylalcohol and terpineol, the hydrocarbons are mainly terpenes and balsams. However, it is preferable to use mixtures of different fragrances which together produce an attractive scent. Essential oils of lesser volatility, which are mostly used as aroma components, are also suitable as perfume oils, e.g. sage oil, camomile oil, clove oil, melissa oil, mint oil, cinnamon leaf oil, lime blossom oil, juniper berry oil, vetiver oil, olibanum oil, galbanum oil, labolanum oil and lavandin oil. Preferably bergamot oil, dihydromyrcenol, lilial, lyral, citronellol, phenylethyl alcohol, α-hexylcinnamaldehyde, geraniol, benzyl acetone, cyclamen aldehyde, linalool, Boisambrene Forte, Ambroxan, indole, hedione, sandelice, citrus oil, mandarin oil, orange oil, allylamylglycolate, cyclovertal, lavandin oil, muscat sage oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzylacetate, rose oxide, romilllat, irotyl and gloramat are used alone or in mixtures.

Possible aromas considered are peppermint oil, spearmint oil, aniseed oil, star aniseed oil, caraway oil, eucalyptus oil, fennel oil, lemon oil, wintergreen oil, clove oil, menthol and the like.

17. Dyes

The substances suitable and approved for cosmetic purposes can be used as dyes, such as, for example, those listed in the publication “Kosmetische Färbemittel” (“Cosmetic dyestuffs”) published by the Farbstoffkomission der Deutschen Forschungsgemeinschaft, Verlag Chemie, Weinheim, 1984, p. 81-106. Examples are cochineal red A (C.I. 16255), patent blue V (C.I.42051), indigotin (C.I.73015), chlorophyllin (C.I.75810), quinoline yellow (C.I.47005), titanium dioxide (C.I.77891), indanthrene blue RS (C.I. 69800) and madder lake (C.I.58000). Luminol may also be contained as a luminescent dye. These dyes are usually used in concentrations of 0.001 to 0.1 wt. %, based on the total mixture.

The total amount of excipients and additives can be 1 to 50, preferably 5 to 40 wt. %, based on the agents. The preparation of the agents can be carried out by usual cold or hot processes; preferably the phase inversion temperature method is used.

The aforementioned substances as well as the claimed and in embodiments described substances to be used in accordance with the invention are not subject to any special exceptional conditions with regard to their size, formation, material selection and technical conception, so that the selection criteria known in the field of application can be applied without restriction.

Further details, features and advantages of the subject-matter of the invention result from the dependent claims and from the following description of the examples, which are purely illustrative and not restrictive.

### EXAMPLE 1

### Reduction of Unpleasant Notes in Soy Sauce Containing Biogenic Amines

For the experiments, a commercially available soy sauce is used which, after analysis, contains 6 ppm phenylethylamine, 1.6 ppm histamine, 1.8 ppm cadaverine and 11.9 ppm putrescine and is regularly rated by the panelists as being too intensively animalistic—fishy, smelly, metallically fermented.

The panelists (n=6-8) rate the attributes animalistic—fishy, metallic as well as woody, sweet, flowery and terpenic, herbal on an unstructured scale from 0 (no taste)—10 (strong taste). The panelists receive the product alone in one sample and the product mixed with a quantity of substances listed in the table in another sample. Thereby, the order is arbitrary and not known by the panelists beforehand. The following table summarizes the results of the examination.

| | | Odor description (indication of relative change | | |
|---|---|---|---|---|
| Soy sauce with biogenic | | in percent) | | |
| amines and an added | Dosage of the | Animalistic- | Woody, | |
| amount of the following | compound in | fishy, | sweet, | Terpenig, |
| compounds | mg/kg | metallic | flowery | herbal |
| Madranol | 1 | −13.9 | 6.6 | −6.0 |
| Beta ionone | 2 | 2.4 | 51.9 | −28.9 |
| Timberol | 1 | −26.5 | 27.9 | −3.8 |
| Cycloionone | 0.5 | 2.3 | 33.8 | 19.6 |
| Alpha ionone | 1 | −20.5 | −4.4 | −7.6 |
| Tabanon | 1 | −18.8 | 13.7 | −8.2 |
| Dihydroionone | 0.5 | 8.9 | 9.5 | −8.3 |
| 5-Methyl-6,7- | 0.5 | −7.4 | −10.0 | −5.2 |
| dihydrocyclopentapyrazine | | | | |
| Beta epoxyionone | 0.5 | −5.4 | 22.8 | 21.1 |

### EXAMPLE 2

### Reduction of Unpleasant Notes of Fish Sauce—Triangle Test

Commercially available fish sauces are used for the experiments and one is selected from these which, after analysis, contains 8 ppm phenylethylamine, 99 ppm histamine, 121 ppm cadaverine and 61 ppm putrescine and which, compared to the other fish sauces, is regarded as too intensely animalistic—fishy, smelly, metallically fermented by the panelists. The panelists received the following instruction: Please smell the three samples and mark the sensorially deviating samples with a cross. In any case, one of the samples must be ticked, even if no difference can be noted.

| Fish sauce with biogenic | | |
|---|---|---|
| amines and an added | Dosage of the | |
| amount of the following | compound in | |
| compounds | mg/kg | Odor description |
| Alpha-Ionol | 0.5 | bready, earthy, rounded |
| Isocyclocitral | 1 | bready, rounded, |
| | | sweet, caramel |
| | | note, hay-like |
| 5-Methyl-6,7- | 0.05 | not fishy, less silage notes, |
| dihydrocyclopentapyrazine | | slightly roasted, earthy |
| 4,6,8- | 0.5 | bready, less |
| Megastigmatrien-3-one | | silage notes, |
| (Tabanon) | | sweeter, less fishy |
| Alpha ionone | 0.1 | bready, earthy, |
| | | rounded, light |
| | | cocoa note |
| 2,3-Diethyl-5-methyl- | 0.1 | metallic, dusty, |
| pyrazine | | |
| Alpha-damascone | 0.2 | cocoa note, sweet, |
| | | mild, not bready |
| Beta ionone | 0.5 | less silage notes, |
| | | bready, mild |

### EXAMPLE 3

### Use of an Aroma Mixture Containing Both Components

Two aroma mixtures A and B with the following composition were used for further experiments:

Aroma Mixture A:

| | | |
|---|---|---|
| α-Ionone | 0.4 | wt. % |
| α-Damascone | 0.6 | wt. % |
| Dimethylpyrazine | 0.2 | wt. % |
| Triglyceride vegetable oil | 98.8 | wt. % |

Aroma Mixture B:

| | | |
|---|---|---|
| β-Damascenone | 0.2 | wt. % |
| 5-Methyl-6,7-dihydro-cyclopentapyrazine | 0.6 | wt. % |
| 5-Methyl-6,7-dihydro-cyclohexen-1-one | 0.8 | wt. % |
| Triglyceride vegetable oil | 98.4 | wt. % |

These aroma mixtures were now added to foodstuffs and, analogous to example 3, evaluated for their odor (each with 5 panelists).

The results are shown in the following table:

| | | | Share | |
|---|---|---|---|---|
| | | | in % | |
| | | | (relative | Sensory |
| | | Aroma | to the | evaluation |
| Sample | | mixture | foodstuff) | (n = 5) |
| 1 | Fish sauce | | | fishy, sweaty, animalistic, |
| | (10% in water) | | | pungent, salty, umami |
| 2 | Fish sauce | A | 0.005 | less fishy, rounder, more |
| | (10% in water) | | | pleasant compared to 1 |
| 3 | Fish sauce | B | 0.001 | less fishy, rounder, umami, |
| | (10% in water) | | | roaster compared to 1 |
| 4 | Dark top- | | | malty, sweet, |
| | fermented | | | bitter, burnt, |
| | beer | | | greasy, spicy |
| 5 | Dark top- | A | 0.01 | malty, less bitter, more |
| | fermented | | | fullness, more pleasant |
| | beer | | | compared to 4 |
| 6 | Dark top- | B | 0.025 | more pleasant, less bitter, |
| | fermented | | | malty, sweeter |
| | beer | | | compared to 4 |
| 7 | Organic | | | sour, fermented, fruity, |
| | sauerkraut | | | carbonaceous, sulfurous, |
| | juice | | | umami |
| 8 | Organic | A | 0.01 | less fermented and |
| | sauerkraut | | | sour than |
| | juice | | | 7, carbonaceous |
| 9 | Organic | B | 0.1 | carbonaceous, sweet, less |
| | sauerkraut | | | fermented and sour compared |
| | juice | | | to 7 |
| 10 | milk with | | | bitter, malty, fruity, |
| | cocoa | | | chocolaty, sweet, creamy |
| 11 | milk with | A | 0.02 | rounder, less bitter, |
| | cocoa | | | chocolaty, sweeter compared |
| | | | | to 10 |
| 12 | milk with | B | 0.005 | rounder, less bitter, |
| | cocoa | | | chocolaty, roasted, sweeter |
| | | | | compared to 10 |
| 13 | Zweigelt | | | astringent, fruity, |
| | red wine | | | berry, bitter |
| 14 | Zweigelt | A | 0.001 | more harmonious, less |
| | red wine | | | astringent, fruity, berry |
| | | | | compared to 13 |
| 15 | Zweigelt | B | 0.005 | more harmonious, less |
| | red wine | | | astringent, fruity, berry |
| | | | | compared to 13 |
| 16 | Pork | | | roasted, animalistic, greasy, |
| | Jerky | | | sulfurous |
| 17 | Pork | A | 0.02 | roasted, sulfurous, greasy, |
| | Jerky | | | balanced |
| 18 | Pork | B | 0.04 | roasted, greasy, sulfurous |
| | Jerky | | | balanced |
| 19 | Liquid special | | | ammoniacal, fishy, |
| | food | | | metallic |
| 20 | Liquid special | A | 0.02 | less pungent ammoniacal, |
| | food | | | fatty-metallic, fishy |
| 21 | Liquid special | B | 0.04 | less ammoniacal and fishy, |
| | food | | | metallic |

One can see very clearly how a combination of the two components has a positive effect on the taste of all samples tested.

The individual combinations of the components and characteristics of the above-mentioned embodiments are exemplary; the exchange and substitution of these teachings with other teachings contained in this publication with the cited publications are also explicitly considered. The skilled person recognizes that variations, modifications and other embodiments described here may also occur without deviating from the inventive idea and the scope of the invention. Accordingly, the above description is to be considered as exemplary and not restrictive. The word ‘comprising’ used in the claims does not exclude other components or steps. The indefinite article “an” does not exclude the meaning of a plural. The mere fact that certain measures are recited in mutually different claims does not emphasize that a combination of those measures cannot be used advantageously. The scope of the invention is defined in the following claims and the corresponding equivalents.

1. An aroma mixture comprising a first component comprising a molecule selected from the group comprising molecules of the following structure (I): 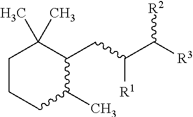
wherein [symbol] represents a single or double bond, wherein, allenes are excluded,
R¹ is selected from hydrogen or methyl,
R² is O or OH, and
R³ is selected from methyl, ethyl, propyl, isopropyl or propenyl,
molecules of the following structure (II): 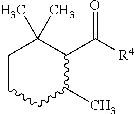
wherein [symbol] represents a single or double bond, wherein allenes or cumulene structures are excluded,
R⁴ is selected from hydrogen and propenyl,
2,5,5,8a-tetramethyl-7,8-dihydro-6H-chromene (cyclo-ionone), 4,4,7a-trimethyl-6,7-dihydro-5H-benzofuran-2-one (dihydroactinidiolide), (E)-4-(1,5,5-trimethyl-7-oxabicyclo[4.1.0]heptan-6-yl)but-3-en-2-one (5,6-epoxy ionone),
2-(2,6,6-trimethylcyclohex-1-en-1-yl) acetaldehyde (β-homocyclocitral), (4E)-4-[(E)-but-2-enylidene]-3,5,5-trimethyl-cyclohex-2-en-1-one (Tabanon), (E)-1-(2,4,4-trimethylcyclohex-2-en-1-yl)but-2-en-1-one (α-isodamascone), and 4,4,7-trimethyl-3,6,7,8-tetrahydronaphthalen-2-one,
or mixtures thereof,
as well as a second component comprising an alkylated 1,4-pyrazine having one to four alkyl carbons.

2. A foodstuff or foodstuff preparation comprising a biogenic amine and the first and second components of claim 1, wherein the first and second components are independently present in the foodstuff or foodstuff preparation in at least a concentration of ≥1 μg/kg.

3. A method of reducing and/or masking the taste and/or odor of biogenic amines comprising,
adding a first component comprising a molecule selected from the group comprising molecules of the following structure (I): 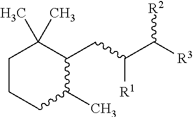
wherein [symbol] represents a single or double bond, wherein, allenes are excluded,
R¹ is selected from hydrogen or methyl,
R² is O or OH, and
R³ is selected from methyl, ethyl, propyl, isopropyl or propenyl,
molecules of the following structure (II): 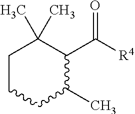
wherein [symbol] represents a single or double bond, wherein allenes or cumulene structures are excluded,
R⁴ is selected from hydrogen and propenyl,
2,5,5,8a-tetramethyl-7,8-dihydro-6H-chromene (cyclo-ionone), 4,4,7a-trimethyl-6,7-dihydro-5H-benzofuran-2-one (dihydroactinidiolide), (E)-4-(1,5,5-trimethyl-7-oxabicyclo[4.1.0]heptan-6-yl)but-3-en-2-one (5,6-epoxy ionone),
2-(2,6,6-trimethylcyclohex-1-en-1-yl) acetaldehyde (β-homocyclocitral), (4E)-4-[(E)-but-2-enylidene]-3,5,5-trimethyl-cyclohex-2-en-1-one (Tabanon), (E)-1-(2,4,4-trimethylcyclohex-2-en-1-yl)but-2-en-1-one (α-isodamascone), and 4,4,7-trimethyl-3,6,7,8-tetrahydronaphthalen-2-one,
or mixtures thereof,
or a second component comprising an alkylated 1,4-pyrazine having one to four alkyl carbons to an aroma mixture.

4. The aroma mixture according to claim 1, wherein the first component comprises at least one molecule selected from the group comprising:
(E)-4-(2,6,6-trimethylcyclohexen-1-yl)but-3-en-2-one (β-ionone), (E)-4-(2,6,6-trimethylcyclohex-2-en-1-yl)but-3-en-2-one (α-ionone), (E)-4-(2,6,6-trimethylcyclohex-2-en-1-yl)but-3-en-2-ol (α-lonol), (E)-4-(2,6,6-trimethylcyclohex-1-en-1-yl)but-3-en-2-ol (β-lonol), 1-(2,2,6-trimethylcyclohexyl)pentan-3-ol (Madranol), 1-(2,2,6-trimethylcyclohexyl) hexan-3-ol (Timberol), 2,5,5,8a-tetramethyl-7,8-dihydro-6H-chromene (cyclo ionone), 4,4,7a-trimethyl-6,7-dihydro-5H-benzofuran-2-one (dihydroactinidiolide), (E)-4-(1,5,5-trimethyl-7-oxabicyclo[4.1.0]heptan-6-yl)but-3-en-2-one (5,6-epoxy-ionone), (E)-1-(2,6,6-trimethylcyclohex-3-en-1-yl)but-2-en-1-one (δ-damascone), (E)-1-(2,6,6-trimethylcyclohex-1-en-1-yl)but-2-en-1-one (β-damascone), (E)-1-(2,6,6-trimethylcyclohex-2-en-1-yl)but-2-en-1-one (α-damascone), (E)-1-(2,6,6-trimethylcyclohexa-1,3-dien-1-yl)but-2-en-1-one (β-damascenone), 2-(2,6,6-trimethylcyclohex-1-en-1-yl) acetaldehyde (β-homocyclocitral), 2,6,6-trimethylcyclohex-1-ene-1-carbaldehyde (β-cyclocitral), (E)-1-(2,4,4-trimethylcyclohex-2-en-1-yl)but-2-en-1-one (α-isodamascone), (4E)-4-[(E)-but-2-enylidene]-3,5,5-trimethyl-cyclohex-2-en-1-one (Tabanon), and 4,4,7-trimethyl-3,6,7,8-tetrahydronaphthalen-2-one, or mixtures thereof.

5. An aroma mixture according to claim 1, wherein the first component comprises at least one molecule selected from the group comprising of 5-methyl-6,7-dihydro-5H-cyclopenta[b]pyrazine, 2,3-diethyl-5-methyl-pyrazine, 2-ethyl-3,5-dimethyl-pyrazine, and 3-ethyl-2,5-dimethyl-pyrazine, or mixtures thereof.

6. A ready-made product containing an aroma mixture according to claim 1.

7. A cosmetic agent containing an aroma mixture according to claim 1.

8. A pharmaceutical preparation for the protection of the human or animal body containing an aroma mixture according to claim 1.

9. The method of claim 3, comprising adding both the first component and the second component to the aroma mixture.

10. The aroma mixture according to claim 4, wherein the first component comprises at least one molecule selected from the group comprising of 5-methyl-6,7-dihydro-5H-cyclopenta[b]pyrazine, 2,3-diethyl-5-methyl-pyrazine, 2-ethyl-3,5-dimethyl-pyrazine, and 3-ethyl-2,5-dimethyl-pyrazine, or mixtures thereof.

11. A ready-made product containing an aroma mixture according to claim 4.

12. A ready-made product containing an aroma mixture according to claim 5.

13. A ready-made product containing a foodstuff according to claim 3.

14. A cosmetic agent containing an aroma mixture according claim 4.

15. A cosmetic agent containing an aroma mixture according to claim 5.

16. A pharmaceutical preparation for the protection of the human or animal body containing an aroma mixture according to claim 4.

17. A pharmaceutical preparation for the protection of the human or animal body containing an aroma mixture according to claim 5.
